(19) United States
(12) Patent Application Publication
(10) Pub. No.: US 2009/0030465 A1
(43) Pub. Date: Jan. 29, 2009

(54) Dynamic rod

(76) Inventors: Moti Altarac, Irvine, CA (US); Joey Camia Reglos, Lake Forest, CA (US); Stanley Kyle Hayes, Mission Viejo, CA (US); Daniel H. Kim, Houston, TX (US)

Correspondence Address: RIMAS LUKAS;VERTIFLEX, INC., 1351 CALLE AVANZADO, SAN CLEMENTE, CA, 92673, US

(21) Appl. No.: 12/233,212

(22) Filed: Sep. 18, 2008

Related U.S. Application Data
(63) Continuation-in-part of application No. 12/154,540, filed on May 23, 2008.
Application No. 12/154,540 is a continuation-in-part of application No. 11/427,738, filed on Jun. 29, 2006.
Application No. 11/427,738 is a continuation-in-part of application No. 11/436,407, filed on May 17, 2006.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
(60) Provisional application No. 60/994,899, filed on Sep. 21, 2007.
(60) Provisional application No. 60/931,811, filed on May 25, 2007.

(51) Int. Cl. A61B 17/70

(52) U.S. Cl. 606/257; 606/264; 606/278

(57) ABSTRACT

A dynamic rod implantable into a patient and connectable between two vertebral anchors in adjacent vertebral bodies is provided. The rod fixes the adjacent vertebral bodies dynamically providing immediate postoperative stability and support of the spine. The rod comprises a first rod portion having a first engaging portion connected to a second rod portion having a second engaging portion. The rod includes at least a one bias element configured to bias a deflection or translation of the first rod portion relative to the second rod portion. The first engaging portion includes at least one side or integral spring formed in the first engaging portion to bias a deflection of the second rod portion relative to the first rod portion. The rod permits relative movement of the first and second rod portions allowing the rod to carry some of the natural flexion and extension moments that the spine is subject to.

### CROSS-REFERENCE TO RELATED APPLICATIONS

This application claims the benefit of and is a continuation-in-part of U.S. Provisional Patent Application Ser. No. 60/994,899 entitled “Dynamic rod” filed on Sep. 21, 2007 which is incorporated herein by reference in its entirety. This application also claims priority to and is a continuation-in-part of co-pending U.S. patent application Ser. No. 12/154,540 entitled “Dynamic rod” filed on May 23, 2008 which is a non-provisional of U.S. Provisional Patent Application Ser. No. 60/931,811 entitled “Dynamic rod” filed on May 25, 2007, all of which are hereby incorporated by reference in their entireties. This application also claims priority to and is a continuation-in-part of co-pending U.S. patent application Ser. No. 11/427,738 entitled “Systems and methods for stabilization of the bone structures” filed on Jun. 29, 2006 which is a continuation-in-part of U.S. patent application Ser. No. 11/436,407 entitled “Systems and methods for stabilization of the bone structures” filed on May 17, 2006 which is a continuation-in-part of U.S. patent application Ser. No. 11/033,452 entitled “Systems and methods for stabilization of the bone structures” filed on Jan. 10, 2005 which is a continuation-in-part of U.S. patent application Ser. No. 11/006,495 entitled “Systems and methods for stabilization of the bone structures” filed on Dec. 6, 2004 which is a continuation-in-part of U.S. patent application Ser. No. 10/970,366 entitled “Systems and methods for stabilization of the bone structures” filed on Oct. 20, 2004. All of the above-referenced applications are each incorporated herein by reference in their entirety.

### FIELD

The present invention generally relates to devices, systems, and methods for the fixation of the spine. In particular, the present invention relates to a rod system applied to the spine that provides dynamic support to spinal vertebrae.

### BACKGROUND

Damage to the spine as a result of advancing age, disease, and injury, has been treated in many instances by fixation or stabilization of vertebrae. Conventional methods of spinal fixation utilize a rigid spinal fixation device to support an injured spinal vertebra relative to an adjacent vertebra and prevent movement of the injured vertebra relative to an adjacent vertebra. These conventional spinal fixation devices include anchor members for fixing to a series of vertebrae of the spine and at least one rigid link element designed to interconnect the anchor members. Typically, the anchor member is a screw and the rigid link element is a rod. The screw is configured to be inserted into the pedicle of a vertebra to a predetermined depth and angle. One end of the rigid link element is connected to an anchor inserted in the pedicle of the upper vertebra and the other end of the rod is connected to an anchor inserted in the pedicle of an adjacent lower vertebra. The rod ends are connected to the anchors via coupling constructs such that the adjacent vertebrae are supported and held apart in a relatively fixed position by the rods. Typically two rods and two pairs of anchors are installed each in the manner described above such that two rods are employed to fix two adjacent vertebrae, with one rod positioned on each side of adjacent vertebrae. Once the system has been assembled and fixed to a series of two or more vertebrae, it constitutes a rigid device preventing the vertebrae from moving relative to one another. This rigidity enables the devices to support all or part of the stresses instead of the stresses being born by the series of damaged vertebra.

While these conventional procedures and devices have been proven capable of providing reliable fixation of the spine, the resulting constructs typically provide a very high degree of rigidity to the operative levels of the spine resulting in decreased mobility of the patient. Unfortunately, this high degree of rigidity imparted to the spine by such devices can sometimes be excessive. Because the patient's fixed vertebrae are not allowed to move, the vertebrae located adjacent to, above or below, the series that has undergone such fixation tend to move more in order to compensate for the decreased mobility. As a result, a concentration of additional mechanical stresses is placed on these adjacent vertebral levels and a sharp discontinuity in the distribution of stresses along the spine can then arise between, for example, the last vertebra of the series and the first free vertebra. This increase in stress can accelerate degeneration of the vertebrae at these adjacent levels.

Sometimes, fixation accompanies a fusion procedure in which bone growth is encouraged to bridge the intervertebral body disc space to thereby fuse adjacent vertebrae together. Fusion involves removal of a damaged intervertebral disc and introduction of an interbody spacer along with bone graft material into the intervertebral disc space. In cases where fixation accompanies fusion, excessively rigid spinal fixation is not helpful to the promotion of the fusion process due to load shielding away from the fixed series. Without the stresses and strains, bone does not have loads to adapt to and as bone loads decrease, the bone becomes weaker. Thus, fixation devices that permit load sharing and assist the bone fusion process are desired in cases where fusion accompanies fixation.

Various improvements to fixation devices such as a link element having a dynamic central portion have been devised. These types of dynamic rods support part of the stresses and help relieve the vertebrae that are overtaxed by fixation. Some dynamic rods are designed to permit axial load transmission substantially along the vertical axis of the spine to prevent load shielding and promote the fusion process. Dynamic rods may also permit a bending moment to be partially transferred by the rod to the fixed series that would otherwise be completely born by vertebrae adjacent to the fixed series. Compression or extension springs can be coiled around the rod for the purpose of providing de-rotation forces as well as relative translational sliding movement along the vertical axis of the spine. Overall, the dynamic rod in the fixation system plays an important role in recreating the biomechanical organization of the functional unit made up of two fixed vertebrae together with the intervertebral disc.

In conclusion, conventional spinal fixation devices have not provided a comprehensive solution to the problems associated with curing spinal diseases in part due to the difficulty of creating a system that mimics a healthy functioning spinal unit. Hence, there is a need for an improved dynamic spinal fixation device that provides a desired level of flexibility to the fixed series of the spinal column, while also providing long-term durability and consistent stabilization of the spinal column.

### SUMMARY

According to one aspect of the invention, a dynamic rod is provided. The dynamic rod includes a first rod portion having a first engaging portion at a first end. The dynamic rod includes a second rod portion having a second engaging portion at a first end. The first and second rod portions connected to each other at the first and second engaging portions and at least one bias element is provided. The at least one bias element is configured to provide a bias force in response to deflection or translation of the second rod portion relative to the first rod portion. Also, at least one side spring is provided and disposed adjacent to the second rod portion to provide a bias force in response to deflection of the second rod portion relative to the first rod portion.

According to another aspect of the invention, a method for dynamically stabilizing a patient's spine is provided. The method includes the step of connecting a first vertebral body and an adjacent second vertebral body with at least one dynamic link element. The at least one dynamic link element comprised of two portions joined together is implanting. And the method includes the step of stabilizing the first vertebral body with respect to the second vertebral body with said at least one dynamic link element having a netural position that is extendable when the patient's spine is flexed forward and does not contract in length from the neutral position when patient's spine is extended backward.

According to another aspect of the invention, a dynamic rod is provided. The dynamic rod includes a first rod portion and a second rod portion connected to the first rod portion such that at least part of the second rod portion is nested inside at least part of the first rod portion. The second rod portion and first rod portion define a neutral longitudinal axis and the second rod portion is deflectable from the neutral longitudinal axis relative to the first rod portion. The dynamic rod further includes at least one bias element formed in the at least part of the first rod portion nesting the second rod portion. The bias element includes a free end and a fixed end and is configured to bias a deflection from the longitudinal axis of the second rod portion relative to the first rod portion wherein the bias is in the direction to return the rod toward the neutral longitudinal axis.

Other advantages will be apparent from the description that follows, including the drawings and claims.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates a perspective view of a dynamic rod according to the present invention.

FIG. 2 illustrates an exploded perspective view of the dynamic rod of FIG. 1 according to the present invention.

FIG. 3 illustrates a cross-sectional view of the dynamic rod of FIG. 1 according to the present invention.

FIG. 4a illustrates a cross-sectional view of a first rod portion of the dynamic rod of FIG. 1 according to the present invention.

FIG. 4b illustrates an end view of a first rod portion of the dynamic rod of FIG. 1 according to the present invention.

FIG. 5 illustrates a perspective view of a second rod portion of the dynamic rod of FIG. 1 according to the present invention.

FIG. 6a illustrates a perspective view of a retainer of the dynamic rod of FIG. 1 according to the present invention.

FIG. 6b illustrates a cross-sectional view of the retainer of FIG. 6a according to the present invention.

FIG. 7a illustrates a side view of a dynamic rod in a contracted state according to the present invention.

FIG. 7b illustrates a side view of a dynamic rod in an extended state according to the present invention.

FIG. 7c illustrates a side view of a dynamic rod in an extended and deflected state according to the present invention.

FIG. 7d illustrates a side view of a dynamic rod in a contracted and deflected state according to the present invention.

FIG. 8 illustrates a cross-sectional view of another variation of a dynamic rod in a contracted state according to the present invention.

FIG. 9a illustrates a cross-sectional view of the dynamic rod of FIG. 8 in a contracted and angled state according to the present invention.

FIG. 9b illustrates a cross-sectional view of the dynamic rod of FIG. 8 in an extended and angled state according to the present invention.

FIG. 10a illustrates a cross-sectional view of another variation of a dynamic rod according to the present invention.

FIG. 10b illustrates a cross-sectional view of another variation of a dynamic rod according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants.

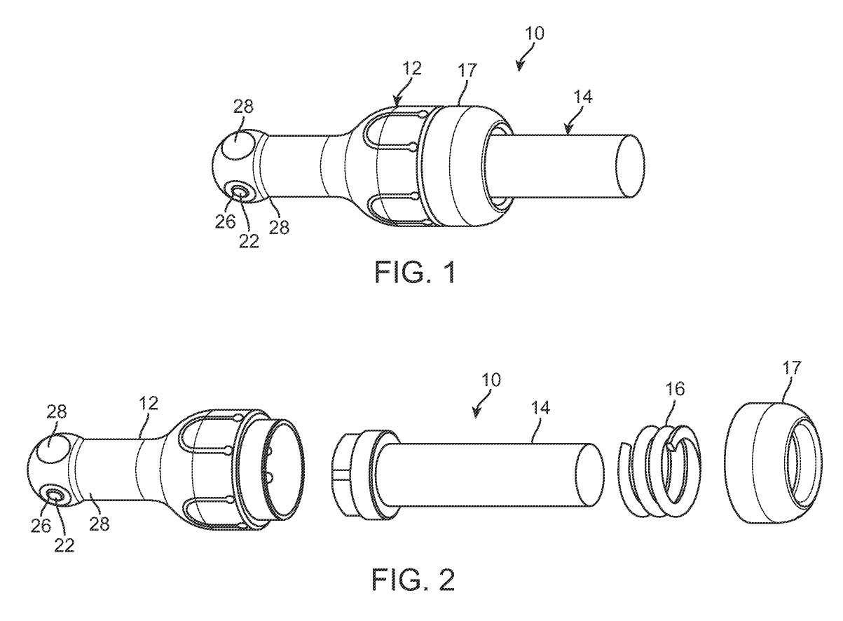

Referring now to FIGS. 1-6, there is shown a dynamic rod 10 for use in a spinal fixation system. A spinal fixation system generally includes a first set of two bone anchor systems installed into the pedicles of a superior vertebral segment, a second set of two bone anchor systems installed into the pedicles of an inferior vertebral segment, a first link element connected between one of the pedicle bone anchor systems in the first set and one of the pedicle bone anchor systems in the second set along the same side of the inferior and superior vertebral segments, and a second link element connected between the other of the pedicle bone anchor systems in the first set and the other of the pedicle bone anchor systems in the second set along the same side of the inferior and superior vertebral segments.

A typical anchor system comprises, but is not limited to, a spinal bone screw that is designed to have one end that inserts threadably into a vertebra and a seat at the opposite end thereof. Typically, the seat is designed to receive the link element in a channel in the seat. The link element is typically a rod or rod-like member. The seat typically has two upstanding arms that are on opposite sides of the channel that receives the rod member. The rod is laid in the open channel which is then closed with a closure member to both capture the rod in the channel and lock it in the seat to prevent relative movement between the seat and the rod.

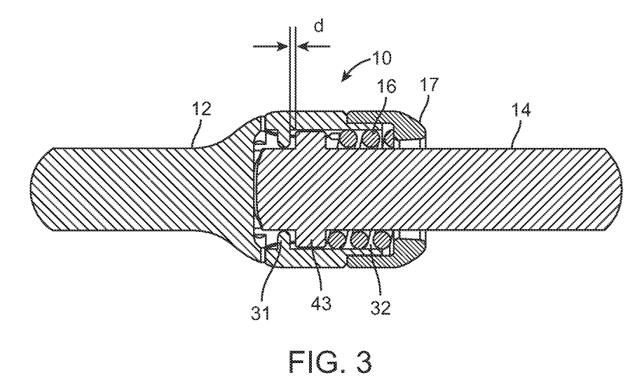

With particular reference to FIGS. 1 and 2, a rod 10 according to the present invention comprises a first rod portion 12, a second rod portion 14, a bias element 16, and a retainer 17 or other connecting means. The first rod portion 12 is connected to the second rod portion 14 via the retainer 17. The bias element 16 is disposed within and between the first and second rod portions 12, 14 as shown in FIG. 3 which illustrates a cross-section of the assembled rod 10.

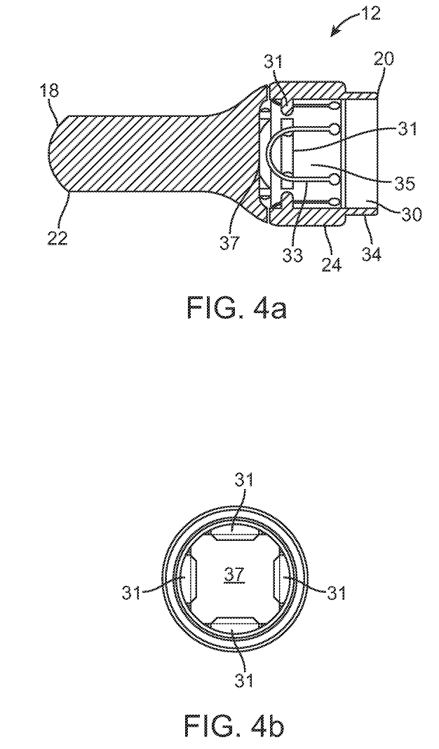

Referring now to FIGS. 4a and 4b, the first rod portion 12 of the dynamic rod 10 will now be described. The first rod portion 12 includes a first end 18 and a second end 20. The first rod portion 12 is generally cylindrical, elongate and rod-like in shape. An anchor connecting portion 22, shown in greater detail in FIGS. 1 and 2, is formed at the first end 18 and configured for attachment to an anchor system. Referring briefly back to FIGS. 1 and 2, the anchor connecting portion 22 is partially spherical in shape and includes oppositely disposed outwardly extending pins 26 for engaging slots formed in the anchor to allow the dynamic rod 10 to pivot about the pins 26 when connected to the anchor. The anchor connecting portion 22 also includes oppositely disposed flat areas 28. When the dynamic rod 10 is connected to the anchor and pivoted into a substantially horizontal position, the flat areas 28 face upwardly and downwardly and as a result, provide a lower profile for the rod within the seat of the anchor. Furthermore, the flat areas 28 provide a flat contact surface for a closure member on the upper surface of the rod and a flat contact surface on the bottom surface when seated in the anchor. Although FIGS. 1 and 2 show the rod having an anchor connecting portion 22 configured for a pin-to-slot engagement, the invention is not so limited and any suitable anchor connecting portion configuration is within the scope of the present invention.

Turning back to FIGS. 4a and 4b, the first rod portion 12 includes an engaging portion 24 at a slightly enlarged and bulbous second end 20. The engaging portion 24 is configured to engage the second rod portion 14 of the dynamic rod 10. The engaging portion 24 includes a first bore defining a receiving portion 30 for receiving the second rod portion 14. The engaging portion 24 also includes at least one abutment or ledge 31 formed within the first bore such that the abutment 31 extends inwardly towards the center of the bore or longitudinal axis. In one variation, the abutment 31 shown in FIG. 4a is divided into four segments as more clearly seen in the end view of FIG. 4b. The engaging portion 24 also includes a least one cut 33 through the engaging portion 24. Although four cuts are shown in FIG. 4a, any suitable number of cuts is within the scope of the invention to create a suitable shape for spring-like effect. The cut 33 is shaped to create a cantilevered spring and in one variation, each cut 33 is substantially U-shaped and forms an integral spring defined in the wall of the engaging portion 24. As a result, the spring is flush with the sidewall of the bore. In the variation with abutments 31, each abutment 31 is located on the inner surface of each spring. The integral spring can also be created by one or more cuts that leave at least a portion of the wall connected to the engaging portion 14 as in a cantilevered leaf spring. The at least one cantilevered side spring has a fixed end and a free end with the fixed end being proximal to the second end 20 of the first rod portion 12 relative to the free such that the springs extends from the fixed end to the free end in a direction towards the first end 18. In another variation, the spring extends from the fixed end to the free end in a direction towards the second end 20 and in another variation extends away from the second end 20. Each cut defines a spring surface 35 that includes each abutment segment 31. Of course, the spring need not be integrally formed but may also be formed by another element disposed laterally of the second rod portion 14 forming a side spring. In the variation shown in FIGS. 4a and 4b, four cuts 33 define four spring surfaces 35 with each spring surface 35 including one abutment segment 31. In particular, the abutment segments 31 are located on the inner spring surface 35 and project inwardly into the first bore. Each abutment segment 31 serves as a contact point for contacting the integral spring with the second rod portion 14. When the second rod portion 14 is deflected relative to the first portion 12, a portion of the second rod portion 14 directly contacts a spring surface 35, and in the variation that includes abutment segments 31, a portion of the second rod portion 14 contacts at least one abutment segment 31 and with sufficient force may deflect the integral spring outwardly creating a bias spring force in a direction that pushes the deflected second rod portion 14 back toward a normal undeflected or neutral-zone position. In one variation, at least three springs are integrally formed and arranged to encompass the engaging portion of the second rod portion. Also, the number of abutments 31 matches the number of side springs. A collar 34 is also formed at the second end 20 that is configured to mate with the retainer 17. The collar 34 has a slightly smaller outer diameter than the rest of the bulbous engaging portion 20. With the retainer 17 mated with the male member collar 34, the intersection of the first rod portion 12 and retainer 17 is substantially flush. A curved base surface 37 is formed at the bottom of the first bore.

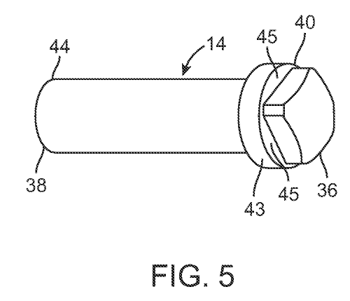

Turning now to FIG. 5, there is shown a second rod portion 14. The second rod portion 14 includes a first end 36 and a second end 38. The second rod portion 14 is generally cylindrical, elongate and rod-like in shape and includes an engaging portion 40 at the first end 36. The engaging portion 40 is configured to engage with the first rod portion 12 of the dynamic rod 10. The engaging portion 40 of the second rod portion 14 includes a spherical or cylindrical feature or collar 43 that allows the second rod portion 14 to angulate inside the first rod portion 12. The first end 36 is shaped to form at least one abutment surface 45 on the collar 43 for contacting the at least one abutment segments 31 of the first rod portion 12. As shown in FIG. 5, four abutment surfaces 45 are formed by a four-sided substantially square first end 36. The substantially square first end 36 controls the rotation of the rod and provides torsional strength and resistance. In general, the first end 36 is shaped to resist torsion of the first rod portion of the first rod portion relative to the second rod portion. At least a portion of the engaging portion 40 of the second rod portion 14 is configured and sized to fit within the receiving portion 30 of the first rod portion 14 as shown in FIG. 3.

Still referencing FIG. 5, the second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor is within the scope of the present invention and, for example, may include a rotatable pin-and-slot or other configuration similar to that shown in FIGS. 1 and 2.

Referring back to FIG. 1, there is shown a bias element 16 according to the present invention. In the variation shown, the bias element 16 is a coil or spring. The bias element 16 is made from any suitable material such as titanium or PEEK. The bias element 16 is sized to be receiving inside the bias element receiving portion 32 between the first rod portion 12 and the second rod portion 14 such that the rod is capable of extension in its length. In particular, the bias element 16 is positioned inside the bias element receiving portion 32 between the collar 43 and the retainer 17 such that, when a force is applied to longitudinally extend the length of the rod, extension is permitted and is biased in the opposite direction by the bias element 16 exerting a spring force to return the second rod portion 14 to its neutral position with respect to the first rod portion 12. Although a coiled spring is shown, the invention is not so limited and any suitable type of bias element may be employed. Different types of biasing elements are discussed in greater detail in related application entitled “Dynamic rod” bearing application Ser. No. 12/154,540 and filed on May 23, 2008 hereby incorporated by reference in its entirety.

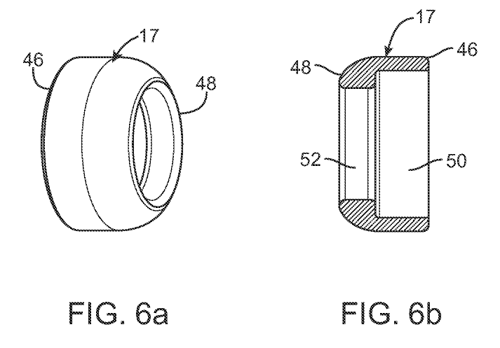

Turning now to FIG. 6, there is shown a retainer 17 having a first end 46 and a second end 48 according to the present invention. The retainer 17 is generally cylindrical and sleeve-like in shape and has a bore opening to and extending between the first and second ends 46, 48. The retainer 17 is configured to encompass at least a portion of the first rod portion 12 and at least a portion of the second rod portion 14 as shown in FIG. 3. Accordingly, the bore defines a first receiving portion 50 at the first end 46 configured to receive therein at least a portion of the first rod portion 12 and, in particular, configured to receive the collar 34 of the first rod portion 12 as shown in FIG. 3. The bore also defines a second receiving portion 52 at the second end 48 that is configured to receive therein at least a portion of the second rod portion. The retainer 17 forms a constriction such that the second end 48 has a smaller diameter relative to the diameter of the retainer 17 at the first end 46. The interior surface of the retainer 17 substantially corresponds to the geometry being received within the retainer 17 with an abutment created at the intersection of the first and second receiving portions 50 and 52 configured to retain the bias element 16.

Referring back to FIGS. 1 and 6, the assembly of the dynamic rod 10 will now be discussed. The bias element 16 is placed over the shaft of the second rod portion 14 from the second end 38. The second rod portion 14 together with the bias element 16 is inserted into the bore of the receiving portion 30 until the collar 43 contacts the at least one abutment 31. The second end 38 of the second rod portion 14 is then inserted into the first end 46 of the retainer 17 and passed through the second end 48 of the retainer 17 such that the collar 34 of the first rod portion 12 is disposed inside the first receiving portion 50 of the retainer 17 and at least a portion of the second rod portion 14 is disposed inside the second receiving portion 52 of the retainer 17. The retainer 17 is connected to the first rod portion 12 by a laser weld or an e-beam weld or other suitable means such that the second rod portion 14 and bias element 16 are captured by the retainer 17 constriction and retained within the retainer 17 and the first rod portion 12 such that the second rod portion 14 is capable of movement relative to the retainer 17 and the first rod portion 12. It is noted that the side springs 35 are located immediately outside the collar 43 such that the collar 43 serves as a deflector of the springs 35. In another variation, the side springs 35 are located immediately outside the engaging portion or first end 36 of the second rod portion. In particular, the second rod portion 14 is capable of displacement from the longitudinal axis and/or movement along the longitudinal axis relative to the retainer 17 and the first rod portion 12. The bias element 16 may also be connected to second rod portion 14 and/or retainer 17 via a laser or e-beam weld.

Movement of the second rod portion 14 relative to the first rod portion 12 along the longitudinal axis such that the rod 10 is moving from a normal or neutral position into extension is biased by the bias element 16. In response to such extension, the bias element 16 exerts a force to return the second rod portion 14 into a normal position. When fully extended from the first rod portion 12, the second rod portion 14 defines a distance “d” between the end of the collar 43 and the abutment 31. This distance “d” defines in part the extent of movement along the longitudinal axis of the second rod portion 14 relative to the first rod portion 12. In one variation, the distance “d” is approximately one or two millimeters. Distance “d” may be customized according to surgeon preference such as upon implantation or be selected to be a suitable distance.

After the dynamic rod 10 is assembled, it is ready to be implanted within a patient and be connected to anchors planted in pedicles of adjacent vertebral bodies preferably in a manner such that the first rod portion 12 of the dynamic rod 10 illustrated in FIGS. 1-2 is oriented cephalad and connected to the upper anchor and the second rod portion 14 is placed caudad and connected to the lower anchor. Because the first rod portion 12 includes an anchor connecting portion 22 configured such that connection with the anchor does not result in the rod extending cephalad beyond the anchor, this orientation and configuration of the dynamic rod is advantageous particularly because it avoids impingement of adjacent anatomy in flexion or in extension of the patient.

In an alternative variation, the dynamic rod 10 is implanted into the patient such that the first rod portion 12 is oriented caudad and the second rod portion 14 is oriented cephalad. In this variation, the second rod portion 14 includes an anchor connecting portion 44 that is partially spherical in shape and includes oppositely disposed outwardly extending pins 54 for engaging slots formed in the upper anchor to allow the dynamic rod 10 to pivot about pins 54 when connected to the anchor. Of course, any connection means is permitted and not limited to a pin-slot engagement. The anchor connecting portion 44 may also include oppositely disposed flat areas 56 as described above. The second rod portion 14 of the dynamic rod 10 is oriented cephalad and connected to the upper anchor and the first rod portion 12 is placed caudad and connected to the lower anchor. Because the second rod portion 14 includes an anchor connecting portion 44 configured such that connection with the anchor does not result in excessive rod extending cephalad beyond the anchor, this orientation and configuration of the dynamic rod is advantageous particularly because it avoids impingement of adjacent anatomy in flexion or in extension of the patient.

Therefore, it is noted that the preferred implantation method and preferred orientation of the dynamic rod 10 is such that there is minimal or substantially no “overhanging” rod extending cephalad beyond the upper anchor. Such orientation is achieved by the orientation of the rod during implantation as well as by the configuration of the anchor connecting portion 22, 44 of either one or both of the first rod portion 12 and second rod portion 14 such that the anchor connecting portion 22, 44 is configured such that there is substantially no overhang beyond the anchor.

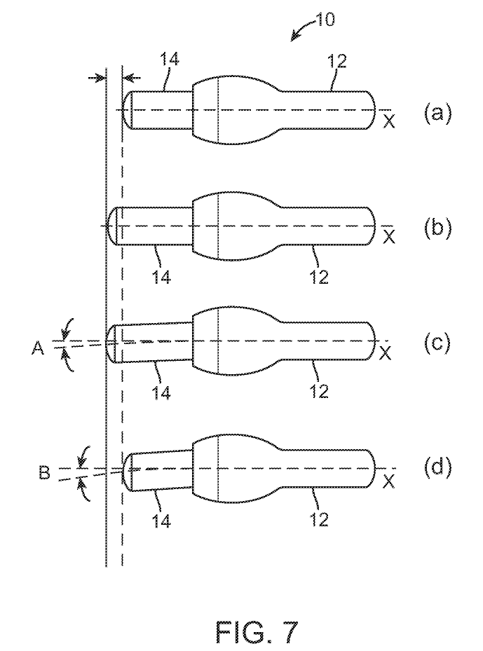

The implanted dynamic rod and anchor system fixes the adjacent vertebral bodies together in a dynamic fashion providing immediate postoperative stability and support of the spine. Referring now to FIG. 7, the dynamic features of the dynamic rod 10 according to the present invention will now be discussed. In FIG. 7a, there is shown a dynamic rod 10 according to the present invention with the second rod portion 14 in a normal position within the first rod portion 12. FIG. 7b shows the second rod portion 14 extended along the longitudinal axis “x” relative to the first rod portion 12. As described above, the degree of longitudinal extension is determined by the configuration of the first and second rod portions 12, 14 and is approximately between zero and five millimeters, preferably approximately one millimeter; however, the invention is not so limited and any suitable longitudinal extension is within the scope of the present invention. FIG. 7c illustrates the second rod portion 14 angled from the longitudinal axis “x” or otherwise displacement of the second rod portion from the longitudinal axis “x” relative to the first rod portion 12 by an angle “A” while the second rod portion 14 is also longitudinally in extension relative to the first rod portion 12. Angle “A” is approximately between zero and five degrees, preferably approximately three degrees with respect to the longitudinal axis “x”. FIG. 7d shows the second rod portion 14 displaced from the longitudinal axis “x” by an angle “B” while in a contracted or normal state. Angle “B” is approximately between zero and five degrees, preferably approximately three degrees with respect to the longitudinal axis “x”.

Hence, FIG. 7 illustrates that the dynamic rod allows for movement described by a displacement from the longitudinal axis as well as movement along the longitudinal axis alone or in combination allowing the rod to carry some of the natural flexion and extension moments that the spine is subjected to. In cases where the dynamic rod is subjected to a force displacing one of the rod portions relative to the other rod portion away from the longitudinal axis, the one or more integral side springs 35 are also displaced from the longitudinal axis. The resulting displacement of the spring surface 35 from the longitudinal axis establishes a biasing force exerted by the integral spring 35 in a direction opposite to its displacement to force the displaced rod portion back into a position substantially aligned with the longitudinal axis. Substantial polyaxial rotation of the second rod portion relative to the first rod portion is within the scope of motion of the dynamic rod. Rotation of the second rod portion 14 relative to the first rod portion 12 is constrained by the squared first end 36 of the second rod portion 14 inserted into a conformance formed by the one or more abutments 31 as seen in FIG. 4. This feature controls rotation and provides torsional strength and resistance.

In one variation, the bias element 16 is a compression spring that becomes shorter when axially loaded and acts as an extension mechanism such that when disposed in the assembled dynamic rod 10 and axially loaded, the bias element 16 exerts a biasing force pushing the first rod portion 12 and the second rod portion 14 apart. In one variation, the bias element 16 is configured such that it exerts a biasing force pushing the first rod portion 12 and second rod portion 14 apart by the maximum degree permitted by the dynamic rod configuration such that when longitudinally loaded the second rod portion 14 will move inwardly towards the first rod portion 12.

The adjacent vertebrae are slightly distracted and the rod is implanted in a patient's spine to relieve pressure on the nerves and offload pressure on the facet and disc. The dynamic rods may be used alone or as an adjunct to fusion. When the rod is implanted in a patient's spine and the patient bends forward, the rod is capable of extending thereby preserving the natural motion of the spine while at the same time offloading pressure and relieving pain. When the patient bends backward, the rod serves as an extension blocker, thereby maintaining the vertebral distraction to relieve pain and assist in reducing impact to allow better load distribution through the level and help prevent adjacent level disease.

In another variation, the bias element 16 is a coil configured to not exhibit spring-like characteristics when loaded along the longitudinal axis. Instead, the coil serves a stabilizer for loads having a lateral force component, in which case the lateral biasing is provided by the bias element.

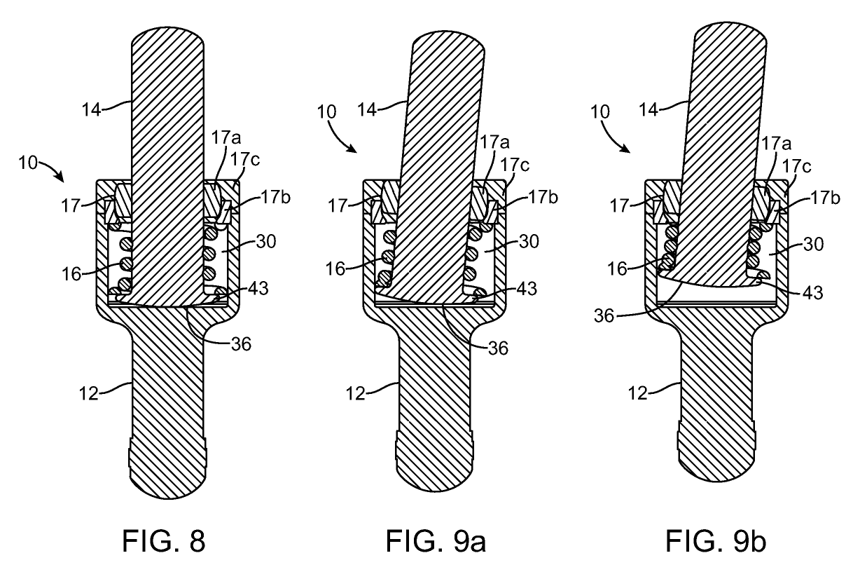

Another dynamic rod 10 according to the present invention is shown in FIGS. 8, 9a and 9b wherein like numbers are used to describe like parts herein. In this variation, the rod 10 includes a first rod portion 12, second rod portion 14, a bias element 16, and a retainer 17. The first rod portion 12 is connected to the second rod portion 14 via the retainer 17 and the bias element 16 is disposed substantially between the first and second rod portions 12, 14 as shown. In this variation, the second rod portion 14 also includes a collar 43 formed at a first end 36 which has a curved or spherical end surface which provides for some rocking motion as shown in FIG. 9a when in contact with the base of the receiving portion 30 of the first rod portion 12.

Still referencing FIGS. 8, 9a and 9b, the retainer 17 includes a ball 17a with a central passageway for the second rod portion 14, a spring retainer 17b and a cap 17c which capture the ball 17a and spring retainer 17b inside the system. The ball 17a is contained with in the retainer 17 and permits displacement from the longitudinal axis as shown in FIGS. 9a and 9b as well as travel along the longitudinal axis as additionally shown in FIG. 9b.

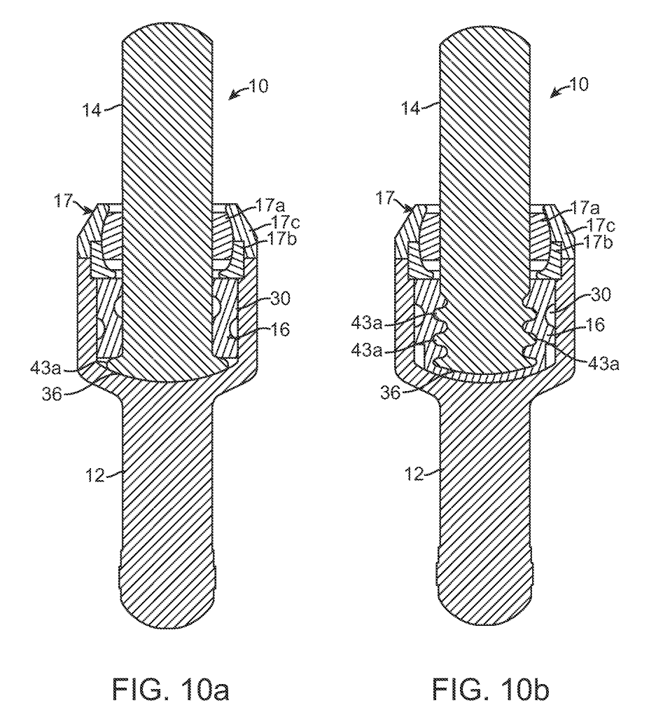

Other dynamic rods 10 according to the present invention is shown in FIGS. 10a and 10b wherein like numbers are used to describe like parts herein. In these variations, the rod 10 includes a first rod portion 12, second rod portion 14, a bias element 16, and a retainer 17. The first rod portion 12 is connected to the second rod portion 14 via the retainer 17 and the bias element 16 is disposed substantially between the first and second rod portions 12, 14 as shown. The second rod portion 14 also includes at least one a ribbed surface 43a at and/or near the first end 36 which has a curved or spherical end surface which provides for some rocking motion as shown in FIG. 9a when in contact with the base of the receiving portion 30 of the first rod portion 12.

Still referencing FIGS. 10a and 10b, the retainer 17 includes a ball 17a with a central passageway for the second rod portion 14, a spring retainer 17b and a cap 17c which capture the ball 17a and spring retainer 17b inside the system. The ball 17a is substantially contained with in the retainer 17 and permits displacement from the longitudinal axis as shown in FIGS. 9a and 9b as well as travel along the longitudinal axis as shown in FIG. 9b. In one variation, the retainer elements 17a, 17b, 17c all made of polymeric materials such that there is no metal-on-metal contact and wear. The bias element 16 in this variation substantially fills the receiving portion 30 surrounding the first end 36 and is molded to conform and grip the second rod portion 14. The bias element 16 can be made of any suitable material such as silicone or PEEK or other polymer and molded separately and then assembled or molded over the second rod portion 14. As shown in FIG. 10b, the bias element 16 in this variation is also disposed between the first end 36 of the second rod portion and the base of the receiving portion 30 of the first rod portion 12 substantially encompassing the first end 36. This variation advantageously additionally dampens the rod under a compression load as well as biasing extension of the second rod portion 14 relative to the first rod portion 12 with a one-piece bias element 16. Therefore, a single bias element both biases a compression load and extension load on the rod 10 with the bias force being in the opposite direction of the load. This variation also prevents metal on metal contact as the end of the second rod portion 14 is substantially enclosed in the bias material. The ribbed surfaces 43a conform to the bias element 16 as shown in FIG. 10b

The dynamic rods 10 of FIGS. 8, 9a, 9b and 10 are implanted into the patient in the same manner as described above with respect to FIGS. 1-7 and fix the adjacent vertebral bodies together in a dynamic fashion. The dynamic rod assembly permits relative movement of the first and second rod portions 12, 14 providing immediate postoperative stability and support of the spine. The dynamic rod allows for movement described by a displacement from the longitudinal axis as well as movement along the longitudinal axis alone or in combination allowing the rod to carry some of the natural flexion and extension moments that the spine is subjected to.

The disclosed devices or any of their components can be made of any biologically adaptable or compatible materials including PEEK, PEK, PAEK, PEKEKK or other polyetherketones. Materials considered acceptable for biological implantation are well known and include, but are not limited to, stainless steel, titanium, tantalum, combination metallic alloys, various plastics, polymers, resins, ceramics, biologically absorbable materials and the like. Any components may be also coated.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A dynamic rod comprising:
a first rod portion having a first engaging portion at a first end;
a second rod portion having a second engaging portion at a first end, the first and second rod portions connected to each other at the first and second engaging portions;
at least one bias element configured to provide a bias force in response to deflection or translation of the second rod portion relative to the first rod portion; and
at least one side spring adjacent to the second rod portion to provide a bias force in response to deflection of the second rod portion relative to the first rod portion.

2. The dynamic rod of claim 1 wherein the at least one side spring is an integral spring formed in the first engaging portion and located adjacent to the first end of the second rod portion.

3. The dynamic rod of claim 2 including more than one side spring arranged to encompass the first end of the second rod portion.

4. The dynamic rod of claim 1 wherein the at least one side spring is cantilevered to the first rod portion; the at least one side spring having a fixed end and a free end with the fixed end being proximate the first end of the first rod portion relative to the free end.

5. The dynamic rod of claim 4, the first rod portion having a second end and wherein the at least one side spring extends from the fixed end to the free end in a direction towards the second end.

6. The dynamic rod of claim 4 wherein each side spring includes an abutment that protrudes toward the second rod portion.

7. The dynamic rod of claim 1 wherein the first and second rod portions are connected to each other at the first and second engaging portions such that the second engaging portion is at least partially inside the first engaging portion.

8. The dynamic rod claim 7 wherein the first and second engaging portions are configured to limit torsion of the first rod portion relative to the second rod portion.

9. The dynamic rod of claim 7 wherein the first bias element is shaped to substantially fill the void between the first engaging portion and the second engaging portion.

10. The dynamic rod of claim 1 wherein the first and second rod portions are configured such that the first bias element exerts a force to return the rod to a normal position when the first rod portion is extended relative to the second rod portion.

11. The dynamic rod of claim 1 wherein the second rod portion further includes a collar and the first bias element is located between the collar and retainer.

12. A method for dynamically stabilizing a patient's spine comprising the steps of:
connecting a first vertebral body and an adjacent second vertebral body with at least one dynamic link element;
implanting at least one dynamic link element comprised of two portions joined together;
stabilizing the first vertebral body with respect to the second vertebral body with said at least one dynamic link element having a netural position that is extendable when the patient's spine is flexed forward and does not contract in length from the neutral position when patient's spine is extended backward.

13. The method of claim 12 further including the step of fusing a third vertebral body of a patient's spine to the second vertebral body; the third vertebral body being adjacent to the second vertebral body.

14. The method of claim 12 further including the step of providing a dynamic link element having a first spring configured to contract the dynamic link element when extended beyond the neutral position.

15. The method of claim 14 further including the step of providing a dynamic link element having a second spring configured to return deflection of one portion with respect to the other portion.

16. A dynamic rod comprising:
a first rod portion;
a second rod portion connected to the first rod portion such that at least part of the second rod portion is nested inside at least part of the first rod portion; said second rod portion and first rod portion defining a neutral longitudinal axis; the second rod portion being deflectable from the neutral longitudinal axis relative to the first rod portion;
at least one bias element formed in the at least part of the first rod portion nesting the second rod portion; the bias element comprising a free end and a fixed end; the bias element configured to bias a deflection from the longitudinal axis of the second rod portion relative to the first rod portion; said bias being in the direction toward the neutral longitudinal axis.

17. The dynamic rod of claim 16 wherein the at least part of the first rod portion nesting the second rod portion encompasses the at least part of the second rod portion.

18. The dynamic rod of claim 17 wherein at least three bias elements are formed in the at least part of the first rod portion nesting the second rod portion and arranged around the at least part of the second rod portion nested inside the first rod portion.

19. The dynamic rod of claim 17 wherein the at least part of the first rod portion nesting the second rod portion has a sidewall and the at least one bias element is defined in the sidewall.

20. The dynamic rod of claim 16 wherein the at least one bias element is cantilevered to the first rod portion at the fixed end.
